(19) United States
(12) Patent Application Publication
(10) Pub. No.: US 2022/0225934 A1
(43) Pub. Date: Jul. 21, 2022

(54) ADJUSTABLE NON-INVASIVE WEARABLE MONITORING DEVICE

(71) Applicant: Wear2B Ltd., Rosh Pina (IL)

(72) Inventors: Ohad BASHAN, Sde Varburg (IL); Giora BAR-SAKAI, Tel Aviv (IL); Oded BASHAN, Tel Aviv (IL)

(73) Assignee: Wear2B Ltd., Rosh Pina (IL)

(21) Appl. No.: 17/614,600

(22) PCT Filed: May 27, 2020
(86) PCT No.: PCT/IL2020/050580

§ 371 Date: Nov. 29, 2021

Related U.S. Application Data
(60) Provisional application No. 62/853,719, filed on May 29, 2019.

(51) Int. Cl. A61B 5/00

(52) U.S. Cl. CPC A61B 5/681; A61B 5/746; A61B 2562/0247; A61B 5/0053; A61B 2562/0261; A61B 5/0059

(57) ABSTRACT

A wearable monitoring device is disclosed. The wearable monitoring device may include: a housing, opened from at least one side; a sensing unit attached to a movable plate; and a controller. The movable plate may be connected to one or more spring-like elements, allowing the movable plate to move with respect to the housing so that when the one or more spring-like elements are extended, the sensing unit protrudes from the open side of the housing and when the one or more spring-like elements are compressed the sensing unit and the movable plate are retracted towards an internal space in the housing.

### FIELD OF THE INVENTION

The present invention generally relates to a non-invasive wearable monitoring device. More particularly, the present invention relates to an adjustable non-invasive wearable monitoring device.

### BACKGROUND OF THE INVENTION

A wearable monitoring device for non-invasive monitoring of a physiological property of a user includes one or more sensors and a wearable element configured to attach the one or more sensors to the body of the user. Most commonly used wearable monitoring devices are smart watches having a heartbeat monitoring sensor and/or a blood oxygen saturation monitoring sensor. In these sensors the user adjusts the smart watches strap according to his/her own personal convenience. Therefore, some users may adjust the sensor on their hand wrist tighter than others.

When measuring physiological properties such as heartbeat and oxygen saturation, the accuracy of the measurements is not sensitive to the amount of tightening of the wearable monitoring devices. However, when using a wearable monitoring device for non-invasive monitoring of other properties, a different approach must be taken. Measuring the chemical composition of the blood (e.g., the sugar levels, cholesterol levels, etc.), electrical conductivity and dehydration of the skin (e.g., measured by bio-impedance sensor), materials in the interstitial fluid such as glucose, and the like may include receiving from the tissue relatively weak measured signals. These week signals may require higher measuring sensitivity. In order to accurately measure a signal indicative of the chemical composition of the blood, or other relatively weak signals, the non-invasive sensor sending and receiving the signal should be attached to the skin of the user while avoiding over-tightening that may affect the blood flow in blood vessels beneath the skin or under-tightening that may affect the accuracy of the measurement.

Therefore, wearable monitoring devices for non-invasive measuring of the chemical composition of the blood may include a mechanism configured to adjust a location of a sensing element of the wearable monitoring device and/or the pressure the sensing element applies on the skin as to avoid over tightening of the device. Furthermore, such a mechanism may further be configured to send an alert to the user's computing device in case of over/under tightening.

### SUMMARY OF THE INVENTION

Some aspects of the invention may be directed to a wearable monitoring device. In some embodiments, the wearable monitoring device may include: a housing, opened from at least one side; a sensing unit attached to a movable plate; and a controller. In some embodiments, the movable plate is connected to one or more spring-like elements, allowing the movable plate to move with respect to the housing so that when the one or more spring-like elements are extended, the sensing unit protrudes from the open side of the housing and when the one or more spring-like elements are compressed the sensing unit and the movable plate are retracted towards an internal space in the housing.

In some embodiments, the wearable monitoring device may further include: at least one displacement sensor for measuring the displacement of the movable plate with respect to a predefined location in the housing. In some embodiments, the controller may be configured to send an alert, to an external computing device, when the displacement is one of: above a first threshold value or under a second threshold value. In some embodiments, a first alert may include instructions for a user to at least one of: adjust the placement of the device and release a tightening of straps included in the device. In some embodiments, a second alert may include instructions for a user to at least one of: adjust the placement of the device, and slightly tightening of straps included in the device.

In some embodiments, the housing is configured to hold the sensing unit and the movable plate. In some embodiments, the wearable monitoring device may further include: an additional housing for holding at least one of: the controller and an additional sensing element. In some embodiments, the housing further holds the controller. In some embodiments, the controller comprises a processor and a communication module for communicating with the external computing device. In some embodiments, the spring-like element is one of: coil spring, volute spring, tension spring, leaf spring and an element made from an elastic material.

In some embodiments, the at least one displacement sensor is at least one of: a sensing element comprising a light source and a light sensor, a force sensing resistor, a strain gauge and a piezoelectric sensor. In some embodiments, the sensing unit may include: at least one light source; and at least one light sensor. In some embodiments, the sensing unit may be configured to apply a predefined pressure on surface of a monitored user's body, wherein the predefined pressure is a function of a spring coefficient of the one or more spring-like units. In some embodiments, the spring coefficient of the one or more spring-like units is selected such that the predefined pressure attaches the sensing unit to the surface of the monitored user's body while avoiding blocking blood flow in blood vessels located beneath the surface.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows an illustration of a wearable monitoring device according to some embodiment of the invention;

FIG. 2 shows an illustration of a wearable monitoring device having a single housing according to some embodiment of the invention;

FIG. 3 shows an illustration of a wearable monitoring device having two housings according to some embodiment of the invention; and

FIG. 4 shows an illustration of an example for displacement sensor according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

Some aspects of the invention may be related to a wearable monitoring device for non-invasively measuring and monitoring of a physiological condition, for example, the chemical composition of the blood. The non-invasive monitoring device may use a sensing unit that may include at least one light emitting source configured to emit light beams towards a user's skin and to measure the returned light with at least one light sensor. In some embodiments, the sensing unit may be coupled to a controller and the returned light may be analyzed in order to determine the level of at least one compound/component (e.g., sugar, cholesterol, etc.) in the blood and/or any layer of the skin.

In some embodiments, to ensure that the sensing element measures the level of at least one compound/component in the blood accurately, the sensing element may by attached to the skin of the user, for example, attached to the inner wrist, while avoiding partial blocking of the blood flow in the blood vessel beneath the skin. Such a partial blocking may be caused by over tightening of the strap(s), holding the wearable monitoring device around the wrist. As used herein, over tightening may be defined as applying pressure on the skin that is greater than the blood pressure in the wrist vessels. Therefore, a non-invasive monitoring device according to embodiments of the invention may include a mechanism that may prevent the over tightening and/or may alert the user if such an over or under tightening occurs.

As oppose to prior art wearable monitoring devices, such as, smart watches, in order to properly work, a devise according to embodiment of the invention may be controllably tightened to the inner wrist. Prior art wearable monitoring devices are configured to work even when loosely attached to the wrist. A user using these prior art wearable monitoring devices can select how tight he/she wants to close the straps of the devise. The smart watches will monitor the user's heartbeats and/or oxygen saturation regardless of the amount of tightening provided by the user.

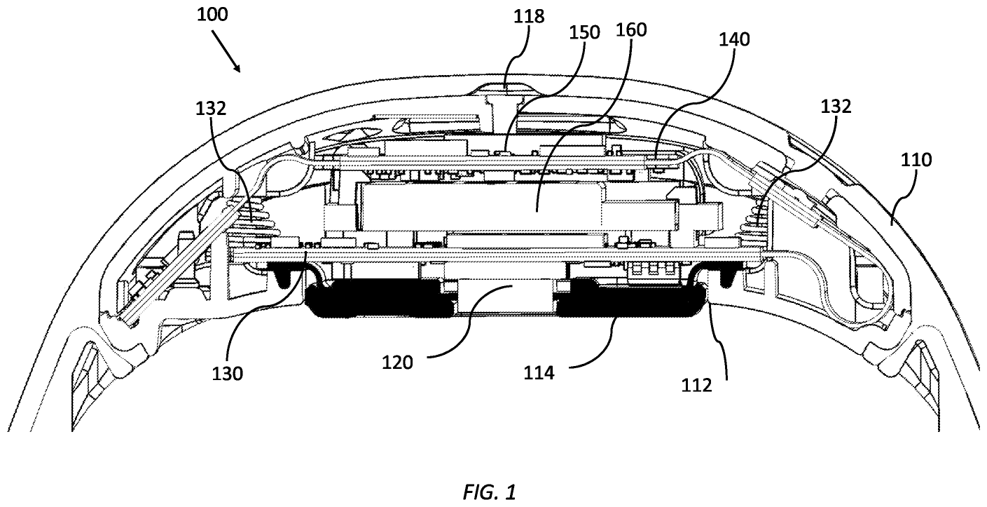

Reference is made to FIG. 1, which is an illustration of a non-invasive monitoring device according to some embodiments of the invention. A non-invasive monitoring device 100 may include a housing 110 opened from at least one side 112, a sensing unit 120 attached to a movable plate 130 and a controller 140 coupled to sensing unit 120. In some embodiments, non-invasive monitoring device 100 may further include a displacement sensor 150 for measuring the displacement of movable plate 130 with respect to a predefined location 118 in housing 110. In some embodiments, device 100 may further include a battery 160 for providing electric power to various components of device 100.

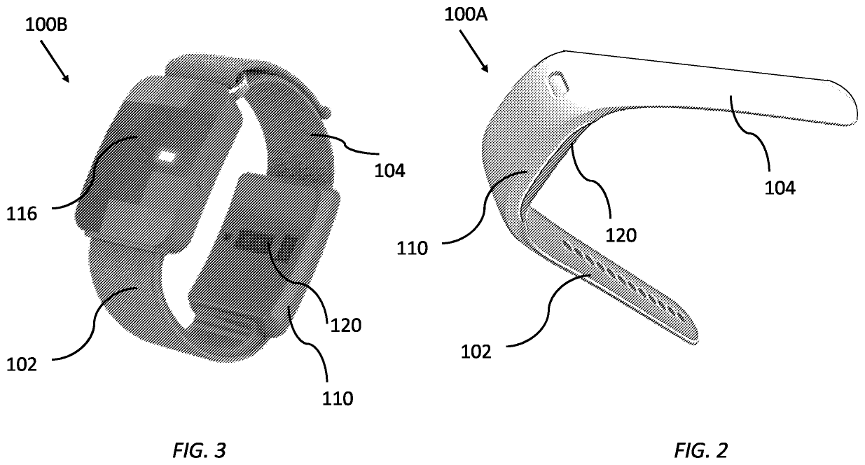

In some embodiments, non-invasive monitoring device 100 may be configured to be worn around the wrist of the user, thus device 100 may further include a strap 102 and/or 104 (illustrated in FIGS. 2 and 3). In some embodiments, housing 110 and straps 102 and 104 may be shaped such that when worn by a user, sensing unit 120 is in contact with the user's skin, for example, sensing unit 120 may be configured to be in contact with the inner wrist of the user, when device 100 is worn by the user.

In some embodiments, sensing unit 120 may include at least one light source, for example, a light emitting diode (LED) configured to emit light at at least one predetermined spectral wavelength band. In some embodiments, sensing unit 120 may include at least one light sensor configured to measure at least one property of the light scattered back from a tissue of the user. The property may be at least one of: the intensity of the scattered light, a phase shift between the emitted and the scattered light, the wavelength of the scattered light and the like.

In some embodiments, movable plate 130 may be or may include any suitable material having stuffiest rigidity as to avoid any flexing of movable plate 130 during the movement of movable plate 130. For example, movable plate 130 may be made of metal (e.g., aluminum alloy, stainless steel, etc.), a rigid polymer (e.g., polystyrene (PS), polypropylene (PP) below the glass transition temperature, and the like).

In some embodiments, movable plate 130 may be connected to one or more spring-like elements 132 allowing the movable plate to move with respect to housing 110 so that when the spring-like elements are extended, sensing unit 120 protrudes from open side 112 of housing 110. In some embodiments, when the one or more spring-like elements 132 are compressed sensing unit 120 and movable plate 130 are retracted towards an internal space in housing 110. In some embodiments, one or more spring like elements 132 may include any element having elastic spring-like properties. For example, one or more spring like elements 132 may be selected from a group consisting of: coil spring, volute spring, tension spring, leaf spring, an element made from an elastic material and the like.

In some embodiments, housing 110 may further include a seal 114 for sealing opening 112, thus protecting sensing element 120 from moisture or any other environmental effects. Seal 114 may be made from any elastic material (e.g., rubber) configured to seal opening 112 and element 120 while allowing sensing element 120 to move with the movement of movable plate 130.

In some embodiments, sensing unit 120 may be configured to apply a predefined pressure on surface (e.g., skin surface) of a monitored user's body (e.g., the skin of the inner wrist). Such predefined pressure may be a function of a spring coefficient of one or more spring-like elements 132. In some embodiments, the spring coefficient of one or more spring-like elements 132 may be selected such that the predefined pressure attaches sensing unit 120 to the surface of the monitored user's body while avoiding blocking blood flow in blood vessels located beneath the surface.

In some embodiments, controller 140 may include a memory and a processor for executing instructions stored in the memory, for example, instructions to control sensing unit 120 to measure a chemical composition of the blood or skin (such as, glucose in the interstitial fluid such as). In some embodiments, controller 140 may further include a communication module for communicating with an external computing device, for example, a user device, such as, a smartphone, a tablet, a laptop and the like.

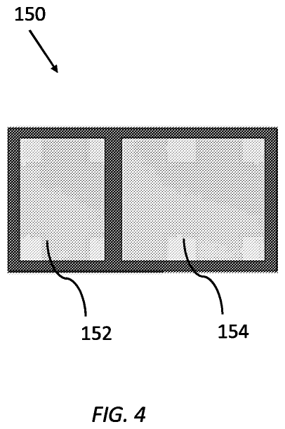

In some embodiments, displacement sensor 150 for measuring the displacement of movable plate 130 with respect to predefined location 118 in housing 110 may include any sensor suitable for detecting such a displacement. In some embodiments, displacement sensor 150 may be attached to movable plate 130. An example, for such a sensor, that includes a light source and a light sensor is given in FIG. 4. Other examples, for such sensor may include one of: a force sensing resistor, a strain gauge, a piezoelectric sensor and the like. In some embodiments, to avoid over-tightening controller 140 may be configured to send an over-tightening alert, to an external computing device (e.g., the user device), when the displacement is above a first threshold value. In some embodiments, to avoid under-tightening (e.g., when spring-like elements are completely loos and movable plate, having for example, a displacement under a second threshold value (e.g., zero)), controller 140 may be configured to send an under-tightening alert.

For example, when one or more spring-like elements 132 are compressed and sensing unit 120 and movable plate 130 are retracted towards an internal space in housing 110, displacement sensor 150 may detect the displacement of movable plate 130. If the displacement is above the threshold value, for example, when one or more spring-like elements 132 are fully compressed, controller 140 may send the first alert to a user device. In some embodiments, when one or more spring-like elements 132 are fully compressed, sensing unit 120 may be over-tightened or over-pressed against the surface of the monitored user's body. In such case, the first alert may include instructions for a user to release the tightening of the device and/or adjust the placement of device 100, for example, by slightly releasing straps 102 and/or 104, illustrated in FIGS. 2 and 3.

In another example, when one or more spring-like elements 132 are almost completely loos and sensing unit 120 and movable plate 130 are extract outward from internal space in housing 110, displacement sensor 150 may detect the displacement of movable plate 130. If the displacement is below a threshold value, for example, zero displacement, controller 160 may sent the second alert that may include instructions for a user to slightly increase the tightening of the device.

Reference is now made to FIG. 2 which is an illustration of a wearable monitoring device having a single housing according to some embodiment of the invention. Housing 110 of a non-invasive wearable monitoring device 100A may be configured to hold sensing unit 120 and movable plate 130 (not illustrated). In some embodiments, housing 110 may further be configured to hold controller 140. In some embodiments, housing 110 of device 100A may be configured to hold all the controllable elements of device 100A, movable plate 130 and one or more spring-like elements 132.

In some embodiments, non-invasive wearable monitoring device 100A may further include straps 102 and/or 104 for holding device 100A around the wrist of the user. In some embodiments, sensing unit 120 may be located in housing 110 as to allow sensing unit 120 to be attached to the inner portion of the wrist of the user when straps 102 and 104 are tightened around the wrist. In some embodiments, housing 110 may further holds battery 160, illustrated in FIG. 1. In some embodiments, housing 110 and straps 102 and/or 104 may be designed to hold device 100A against other parts of the user's body.

Reference is now made to FIG. 3 which is an illustration of a wearable monitoring device having two housings according to some embodiment of the invention. A non-invasive wearable monitoring device 100B may include a first housing 110 for holding sensing unit 120 and movable plate 130 (not illustrated) and a second housing 116 for holding controller 140 and battery 160 (not illustrated). In some embodiments, second housing 116 may further hold another sensing element (not illustrated) that may be similar to sensing element 120 or may be different from sensing element 120. In some embodiments, the other sensing element may be attached to another movable plate 130 that may be connected to additional one or more spring-like elements 132 allowing the other movable plate to move with respect to housing 116. In some embodiments, second housing 116 may further include another displacement sensor 150.

In some embodiments, first strap 102 may connect housing 110 to housing 116 as to form a single inseparable element. In some embodiments, first strap 102 may include internal communication lines and power lines for connecting the electronic components of housing 110 and the electronic components housing 116 to each other, for example, connecting controller 130 and battery 160 to sensing unit 120 and displacement sensor 150. In some embodiments, sensing unit 120 may be located in housing 110 as to allow sensing unit 120 to be attached to the inner portion of the wrist of the user when straps 102 and 104 are tightened around the wrist. In some embodiments, housings 110 and 116 and straps 102 and/or 104 may be designed to hold device 100B against other parts of the user's body.

Reference is now made to FIG. 4 which is an illustration of an example of displacement sensor 150. Displacement sensor 150 may include a light source 152 and a light sensor 154 both being attached to movable plate 130, illustrated in FIG. 1. When assembled in housing 110, illustrated in FIG. 1, light source 152 may emit light towards location 118 in housing 110, Location 118 (illustrated in FIG. 1) may be a location opposite to the opening 112 in housing 110. In some embodiments, light sensor 154 may receive back light reflected from location 118. In some embodiments, controller 140 may calculate the displacement (e.g., the distance or change in distance) of movable plate 130 from location 118, as disclosed, for example, with respect to the disclosure of FIG. 4. In some embodiments, measuring the displacement of movable plate 130 may use as an indirect way for measuring the pressure applied to sensing element 120 (e.g., by user's body surface).

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A wearable monitoring device comprising:
a housing, opened from at least one side;
a sensing unit attached to a movable plate; and
a controller,
wherein the movable plate is connected to
one or more spring-like elements, allowing the movable plate to move with respect to the housing so that when the one or more spring-like elements are extended, the sensing unit protrudes from the open side of the housing and when the one or more spring-like elements are compressed the sensing unit and the movable plate are retracted towards an internal space in the housing.

2. The wearable monitoring device of claim 1, further comprising:
at least one displacement sensor for measuring the displacement of the movable plate with respect to a predefined location in the housing,
and wherein the controller is configured to send an alert, to an external computing device, when the displacement is one of above a first threshold value or under a second threshold value.

3. The wearable monitoring device of claim 2, wherein a first alert includes instructions for a user to at least one of adjust the placement of the device and release a tightening of straps included in the device.

4. The wearable monitoring device of claim 2, wherein a second alert includes instructions for a user to at least one of adjust the placement of the device, and slightly tightening of straps included in the device.

5. The wearable monitoring device of claim 1, wherein the housing is configured to hold the sensing unit and the movable plate.

6. The wearable monitoring device of claim 1, comprising:
an additional housing for holding at least one of: the controller and an additional sensing element.

7. The wearable monitoring device of claim 5, wherein the housing further holds the controller.

8. The wearable monitoring device of claim 1, wherein the controller comprises a processor and a communication module for communicating with the external computing device.

9. The wearable monitoring device of claim 1, wherein the spring-like element is one of: coil spring, volute spring, tension spring, leaf spring and an element made from an elastic material.

10. The wearable monitoring device of claim 2, wherein the at least one displacement sensor is at least one of a sensing element comprising a light source and a light sensor, a force sensing resistor, a strain gauge and a piezoelectric sensor.

11. The wearable monitoring device of claim 1,
wherein the sensing unit comprises:
at least one light source; and
at least one light sensor.

12. The wearable monitoring device of claim 1, wherein the sensing unit is configured to apply a predefined pressure on surface of a monitored user's body, wherein the predefined pressure is a function of a spring coefficient of the one or more spring-like units.

13. The wearable monitoring device of claim 12, wherein the spring coefficient of the one or more spring-like units is selected such that the predefined pressure attaches the sensing unit to the surface of the monitored user's body while avoiding blocking blood flow in blood vessels located beneath the surface.
